(19) United States
(12) Patent Application Publication
(10) Pub. No.: US 2006/0009783 A1
(43) Pub. Date: Jan. 12, 2006

(54) Tunneler with gripping mechanisms

(76) Inventors: Guy Rome, West Valley City, UT (US); Timothy L. Martin, Park City, UT (US); Charles L. Farnworth, Riverton, UT (US)

Correspondence Address: MORRISON & FOERSTER, LLP, 555 WEST FIFTH STREET, SUITE 3500, LOS ANGELES, CA, 90013-1024, US

(21) Appl. No.: 10/888,817

(22) Filed: Jul. 8, 2004

(51) Int. Cl. A61F 11/00

(52) U.S. Cl. 606/108

(57) ABSTRACT

A tissue tunneling device having a gripping mechanism for holding onto the body of the catheter. In one variation, the tissue tunneling device includes an elongate tunneler with a collet attached to the proximal end of the tunneler, and an oversleeve for providing compression to the collet to secure a distal or proximal portion of the catheter within the proximal end of the tunneling device. Internal ribs or surface profiles may be provided on an inner surface of the collet to prevent the catheter from moving when the collet is compressed onto the body of the catheter. The oversleeve may be configured such that over-sliding in the proximal direction is prevented. The collet may further be configured to receive the tips of a split-tip dialysis catheter.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A COMPACT DISK APPENDIX

Not applicable.

### BACKGROUND OF THE INVENTION

In various medical applications an implanted catheter is needed to access a patient's circulatory system. The implanted catheter may be utilized for delivery of medication/fluids or retrieval/sampling of blood. For example, it may be desirable to establish a central line (i.e., access to a large vein) for infusion of medications, chemotherapy drugs, antibiotics, anti-nausea medications, blood products, nutrients or fluids. Implanted catheters are also used in dialysis, apheresis, and other applications requiring diversion of a part of the blood flow in the circulatory system for processing or filtering. However, a common problem associated with implanted catheters is the increased risk of infection due to the establishment of this artificial path into the patient's body. The risk of infection increases the longer the catheter remains implanted.

One common approach to decrease the risk of infection is to “tunnel” the proximal end (i.e., the physician access end) of the catheter within the patient's body such that the catheter enters the body at a location that is displaced from the location that that the catheter enters a major blood vessel within the patient's body. For example, a central line may be established by inserting a catheter into the subclavian vein that runs behind the clavicle, but the catheter entry point into the patient's body may be moved away from an area next to the clavicle to an area that is not immediately above the entry point into the subclavian vein. In this process, the actual access to the subclavian vein is still achieved by puncture under the clavicle, but the proximal portion of the catheter is pulled under the skin for 2-4 inches and emerges from the body at a location close to the nipple. This procedure may allow the catheter to stay in place for weeks to months, or even, in some circumstances, for years.

A tunneler may be utilized to assist in the tunneling of a catheter from a surface entry location on the patient's body to a location where the catheter actually enters a vessel into the circulatory system. Such a tunneler is generally made of steel or hard plastic and has a tapered distal end for tunneling through bodily tissue. Typically, the proximal end of the tunneler has a barb for insertion into the lumen of the catheter. In one application, the catheter placement is accomplished by first making a cut-down incision near the neck of the patient, and then making an exit site incision remote from the cut-down incision. The catheter is attached to the tunneler by forcing the proximal barbed end thereof into the lumen of the catheter and sliding a sleeve over the catheter. A sleeve with a larger diameter may be used so that when the tunnel is made, the hole created is sufficiently large to prevent constricting forces from acting on the catheter as it resides in the tunnel. The tunneler with catheter attached is then pushed from the exit site incision toward the cut-down incision, creating a subcutaneous tunnel. When the tip of the tunneler emerges at the cut-down site, it is grasped by the physician, who pulls it through until reaching the sleeve on the tunneler. The sleeve is then pushed off the catheter and the proximal end of the tunneler is pulled out from the catheter lumen. The catheter distal end is then placed into the blood vessel.

Examples of various tunneling and gripping devices are disclosed in U.S. Patent Application Pub. No. US 2004/0006329 A1, titled “DEVICE FOR HOLDING AND GUIDING A GUIDE WIRE IN A CATHETER” by Scheu, published Jan. 8, 2004; U.S. Patent Application Pub. No. US 2004/0039372 A1, titled “OVER-THE-WIRE CATHETER HAVING A SLIDABLE INSTRUCMENT FOR GRIPPING A GUIDEWIRE” by Carmody, published Feb. 26, 2004; U.S. Pat. No. 3,724,882, titled “TUBE-TO-HOSE CONNECTION” issued to Dehar, dated Apr. 3, 1973; U.S. Pat. No. 4,143,893, titled “CLAMPING DEVICE” issued to Fleischer, dated Mar. 13, 1979; U.S. Pat. No. 4,672,979, titled “SUTURE SLEEVE ASSEMBLY” issued to Pohndorf, dated Jun. 16, 1987; U.S. Pat. No. 5,306,240, titled “TUNNELER AND METHOD FOR IMPLANTING SUBCUTANEOUS VASCULAR ACCESS GRAFTS” issued to Berry, dated Apr. 26, 1994; U.S. Pat. No. 5,405,329, titled “INTRAVASCULAR MULTI-LUMEN CATHETER, CAPABLE OF BEING IMPLANTED BY “TUNNELING”” issued to Durand, dated Apr. 11, 1995; U.S. Pat. No. 6,475,244 B2, titled “TUNNELING DEVICE” issued to Herweck et al., dated Nov. 5, 2002; each of which is incorporated herein by reference in its entirety.

One of the disadvantages of the current tunneling devices is that the tunneler must be differently sized for differently sized lumens, as well as the fact that attachment and removal of the barbed end from the lumen of the catheter often results in damage to the lumen and the distal tip of the catheter. Typically, the barb is larger than the diameter of the catheter lumen and thus forces the tip of the catheter to expand radially and makes the tip prone to damage. Another disadvantage is that the barb attachment mechanism of the tunneler may not be suitable for some of the multi-lumen catheters on the market. For example, some of the multi-lumen catheters have raised profiles at the distal lumen openings which may cause abrasion or resistance when it is passed through the bodily tissue. In addition, some catheters have two or more distal tips. Since the traditional tunnelers are designed to secure only one distal tip, it may be difficult to ensure that all of the tips tunnel through the bodily tissue in a proper manner. Furthermore, some of the catheters in the market do not have a distal opening (e.g. Groshong catheter, etc.), preventing the use of a barbed tunneler on the distal end of such a catheter.

Thus, an improved tunneler capable of accommodating various tip configurations and minimizing damage to the catheter may be desirable. It may be preferable to apply gripping/compression forces for securing the catheter to the tunneler throughout a region or portion of the catheter body instead of limiting/focusing the compression force on a small end portion of the catheter. In addition, it may also be desirable to be able to accommodate catheters with a split-tip design, which have an arterial and venous lumen that bifurcates into two separate branches at the distal end of the catheter.

### BRIEF SUMMARY OF THE INVENTION

Accordingly, described herein is a tunneling device having a gripping mechanism to secure the catheter by holding onto the body of the catheter (i.e., a distal portion or a proximal portion of the catheter) rather than just the distal tip or proximal end of the catheter. This improved tunneler may prevent damage to the catheter that can be experienced using previous designs that require insertion of a barb(s) or other large protrusion(s) into the distal or proximal lumen of the catheter. In one variation, the tunneling device comprises three parts: a tunneler, a collet and a sheath. The tunneler comprises an elongated body with a tapered distal end for insertion into bodily tissues. The tunneler may be fabricated from surgical steel or other metallic and/or polymeric materials with proper strength to support forces needed to separate tissues such that the tunneler may pass through. The collet may comprise a compression sleeve having a plurality of fingers that are separated and flare outward from a unitary distal end away from the central axis of the device. The collet may be configured for positioning at the proximal end of the tunneler. The compression sleeve may be fabricated from metal, plastic or other polymeric material. The sheath may comprise an oversleeve that can be slid over the tunneler from its distal end toward its proximal end to force the fingers on the collet to compress inward toward the central axis of the tunneler.

To assemble, the compression sleeve is slid over the distal end of the tunneler and advanced toward the proximal end. The flared proximal end of the tunneler may have a greater diameter than the inner lumen of the compression sleeve so that the compression sleeve cannot slide off in a proximal direction. The oversleeve is then slid onto the body of the tunneler. To attach a catheter, either the distal or proximal portion of the catheter is placed within the compression sleeve fingers. The oversleeve is then slid over both the catheter and compression sleeve, compressing the combination. The compression sleeve may have internal ribs to further keep the catheter from movement. The oversleeve may also be dimensioned so that sliding too far in the proximal direction is prevented.

The gripper design implemented at the proximal end of the tunneler may provide various advantages, including but not limited to: 1) tunneling forces may be applied to a region along the shaft of the catheter; 2) compression and tensile forces previously required to attach and detach the catheter from the tunneler may be eliminated; 3) tunneler/catheter interface need not depend on the free space within the lumen of the catheter, nor the specific dimensions and geometry thereof as there is no need to insert a barb or other connection mechanism therein to secure the catheter onto the tunneler; and 4) both arterial and venous tips of a split-tip style catheter may be secured within the sheath by the collet, preventing damage thereof during the tunneling process.

These and other embodiments, features and advantages of the present invention will become more apparent to those skilled in the art when taken with reference to the following more detailed description of the invention in conjunction with the accompanying drawings that are first briefly described.

### BRIEF DESCRIPTION OF THE DRAWINGS

FIG. 1A illustrates one variation of a tunneler with a gripping mechanism.

FIG. 1B illustrates an oversleeve for the corresponding tunneler shown in FIG. 1A. The oversleeve may be slidably positioned on the tunnler for compressing the fingers at the proximal portion of the device.

FIG. 1C illustrates a cross sectional view of the tunneler shown in FIG. 1A with a catheter tubing secured between the proximal fingers. The oversleeve is slid over the fingers forcing the fingers to compress inward toward the central axis of the tunneler.

FIG. 2A shows the side view of another variation of a tunneling device with a gripping mechanism. The tunneler is shown with its corresponding oversleeve slidably positioned over the mid-shaft of the tunneler.

FIG. 2B illustrates the shape and dimensions of the compression fingers for the tunneling device shown in FIG. 2A.

FIG. 2C illustrates the compression fingers of FIG. 2B with the compression fingers in the relaxed non-compressed position. As shown, in this variation, the compression fingers expand away from the central axis of the device in the relaxed, non-compressed condition.

FIG. 2D illustrates the oversleeve from FIG. 2A without its corresponding elongated tunneler.

FIG. 2E illustrates the compression of the compression fingers due to the placement of the oversleeve around the compression fingers. As shown, the catheter which is placed between the compression fingers is compressed and secured to the proximal end of the tunneling device.

FIG. 3A-3C illustrates the three separated parts that form another variation of a tunneling device. FIG. 3A shows the tunneler of the tunneling device.

FIG. 3B shows the collet of the tunneling device. The collet may be slid onto the tunneler.

FIG. 3C shows the sheath of the tunneling device. The sheath may be slid onto the tunneler and over the collet to compress the elongated fingers at the proximal end of the collet.

FIG. 3D illustrates the integration of the three parts shown in FIG. 3A-3C for securing a catheter within the proximal portion of the tunneling device. The fingers of the collet are shown in the opened position. A catheter to be attached to the tunneling device is also shown.

FIG. 4A illustrates another variation of the tunneling device. In this variation, the fingers from the compression sleeve are configured to receive a catheter having staggered dual lumens.

FIG. 4B shows the compression sleeve from FIG. 4A detached from the body of the tunneler and having its fingers in the expanded position.

FIG. 4C illustrates the tunneling device of FIG. 4A with a dual lumen catheter placed between the fingers of the compression sleeve. The sheath of the tunneling device is shown in the released position allowing the compression sleeve's fingers to relax and expand outward way from the catheter.

FIG. 4D illustrates the tunneling device of FIG. 4C where the sheath is slid onto the compression sleeve forcing the fingers to compress inward and securing the distal portion of the catheter within the proximal end of the tunneling device. As shown in this cross-sectional view, the distal lumens of the catheters are protected within the tunneling device.

FIG. 4E is an inset figure from FIG. 4D, showing an expanded view of the proximal portion of the device. As shown, ribs are provided on the inner surface of the fingers for securing the catheter tubing within the device.

FIG. 5 illustrates another variation of the tunneling device where the oversleeve and the shaft of the tunneler are configured with matching helical grooves, which allows incremental advancement of the oversleeve on the shaft by the rotation of the oversleeve.

FIG. 6 illustrates another variation of the tunneling device where the proximal end of the tunneler further comprises a rod for insertion into the lumen of the catheter to prevent the collapse of the catheter due to the compression force.

FIG. 7 is a semi-transparent view illustrating anther variation of the tunneling device wherein the oversleeve is configured with a helical ribbon position within the inner lumen of the oversleeve. The ribbon may be configured to provide direct compression on the catheter placed within the oversleeve and, in the process, secures either the distal portion or the proximal portion of the catheter within the oversleeve.

### DETAILED DESCRIPTION OF THE INVENTION

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Before describing the present invention, it is to be understood that unless otherwise indicated this invention need not be limited to applications in human. As one of ordinary skill in the art would appreciate, variations of the invention may be applied to other mammals as well. Moreover, it should be understood that embodiments of the present invention may be applied in combination with various catheters, tubing or other elongated and flexible material/device/tissues/organs for insertion of such material/device/tissues/organs into a patient's body.

Furthermore, although variations of the tunneling device disclosed herein may be particularly usefully for gripping the distal portion of a catheter having a special tip design, it is to be understood that the application of the device is not limited procedures requiring gripping of the catheter from the distal end. One of ordinary skill in the art, having the benefit of the disclosures herein, would appreciate that variations of the tunneling device may be used for gripping the proximal portion of a catheter for tunneling into or under tissues.

A hemodialysis catheter implantation is used herein as an example application of the tunneling device to illustrate the various aspects of the invention disclosed herein. In light of the disclosure herein, one of ordinary skill in the art would appreciate that the tunneling device may be applicable for tunneling various elongated instruments or materials through the soft tissues of a patient. It is also contemplated that the tunneling device described herein may be used for tunneling vascular grafts or other harvested or processed tissues or organs through the muscles or other soft tissues in a patient's body. The tunneling device may also be implemented for inserting elongated instruments, materials, or soft tissues through a hollow body organ in a patient's body.

It must also be noted that, as used in this specification and the appended claims, the singular forms “a,” “an” and “the” include plural referents unless the context clearly dictates otherwise. Thus, for example, the term “a finger” is intended to mean a single finger or a combination of fingers, “a fluid” is intended to mean one or more fluids, or a mixture thereof.

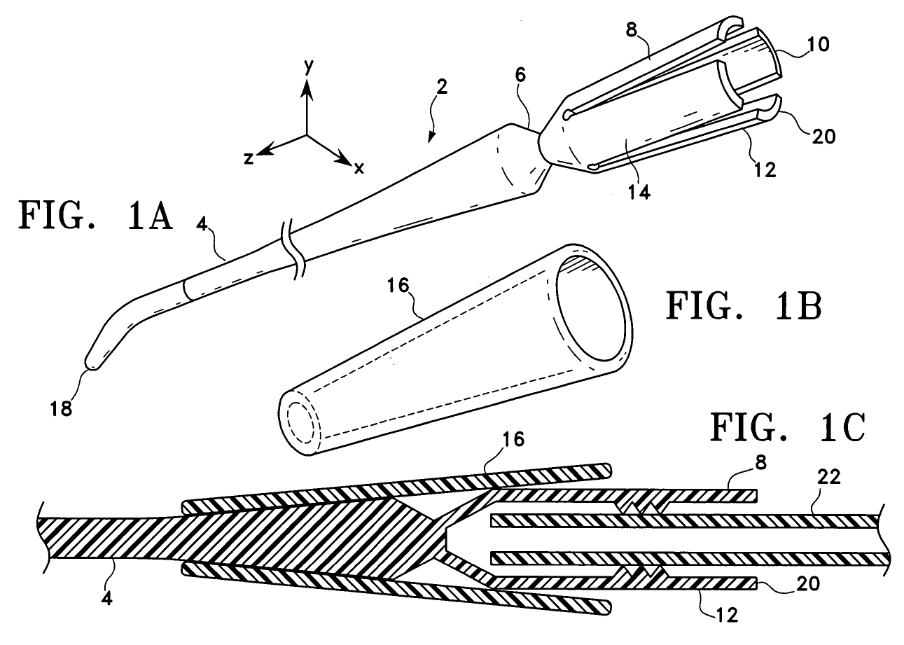

In one aspect of the invention, the tunneling device comprises an elongated body for tunneling through tissues in a patient, and a gripping mechanism attached to the proximal end of the elongated body for securing either the distal portion or the proximal portion of a catheter within the proximal end of the tunneling device. The gripping mechanism may further comprise a compression mechanism to secure the distal/proximal portion of a catheter or other elongated body within the gripping mechanism. For example, a compression mechanism, such as an oversleeve, may be provided to maintain the compression pressure on the body of the catheter, and thus, prevent the catheter from prematurely detaching from the tunneling device. The distal direction of the tunneler is defined as the direction towards the distal end of the tunneler (e.g., +Z in FIG. 1A). Generally, the distal end of the tunneler is configured for insertion into an orifice or opening in a tissue or organ. The distal end the tunneler may also be forced into a tissue to create an opening/channel for tunneling. The proximal direction of the tunneler is defined as the direction toward the proximal end of the catheter (e.g., −Z in FIG. 1A).

In one variation, as shown in FIG. 1A, the tunneling device 2 comprises an elongated body forming a tunneler 4. At the proximal end 6 of the tunneler 4, fingers 8, 10, 12, 14 extending toward the proximal direction are provided for grabbing a catheter or other elongated body. The body of the tunneler 4 may be comprised of metal, metal alloy, polymer or a combination thereof. The fingers 8, 10, 12, 14 extending from the proximal end 6 of the tunneler 4 may be comprised of metal, metal alloy, polymer or a combination thereof. One may fabricate the elongated body with all metal or all polymeric materials; alternatively, one may fabricate the fingers and the elongated shaft of the tunneler with different materials. For example, the shaft may be comprised of metal while the fingers may be comprised of polymer. In another variation, the fingers may be comprised of metallic material while the shaft may be comprised of polymeric material.

The fingers are configured to receive the distal or proximal portion of a catheter, and secure that portion of the catheter within the fingers such that the tunneler may be used to pull the catheter through an orifice within a patient's body. In light of the disclosure herein, one of ordinary skill in the art would appreciate that the proximal end of the tunneler may be configured with other similar mechanical mechanisms that are well known to one of ordinary skill in the art to receive and secure portion of a catheter. For example, clamps or springs may be used to provide the gripping mechanism to trap the distal or proximal portion of a catheter. Furthermore, although the example shown in FIG. 1A has four fingers, one or ordinary skill in the art would appreciate that the device may be configured with two, three, or more fingers.

The tunneling device 2 shown in FIG. 1A further comprises an oversleeve 16, as shown in FIG. 1B, which can be slidably positioned on the shaft of the tunneler 4. The oversleeve 16 may be slid onto the tunneler from the distal end 18 of the tunneler and slid towards the proximal end 20 of the tunneler. As the oversleeve reaches the fingers 8, 10, 12, 14, the fingers will be compressed inward toward the central axis of the tunneling device 2. FIG. 1C illustrates a catheter 22 which has been placed between the fingers 8, 10, 12, 14 at the proximal end 20 of the tunneler. As shown, the oversleeve 16 is pushed over the fingers 8, 10, 12, 14, forcing the fingers to compress inward, trapping portion of the catheter 22. The oversleeve 16 may be comprised of metal, metal alloy, polymeric material, or a combination thereof. Preferably, the fingers 8, 10, 12, 14 are configured such that when relaxed (i.e., in a non-compressed state), they will expand outward away from the axis of the tunneler. In this variation, the fingers will compress inward, trapping the catheter in place when the oversleeve is slid over a portion of the fingers, forcing the fingers to collapse toward the center axis of the tunneler. Although in this variation an oversleeve is implemented to provide the compression, other compression mechanisms that are well known to one of ordinary skill in the art may also be implemented to secure portion of the catheter body within the tunneling device. In addition, the fingers at the proximal end of the tunneler may be configured to receive catheters of various sizes. For example, the space between the fingers may be selected such that it is large enough to receive a 26 French catheter, while at the same time the fingers may be compressed far enough to secure a 6 French catheter. This may allow the physician to utilize the same tunneling device to tunnel catheters of various sizes.

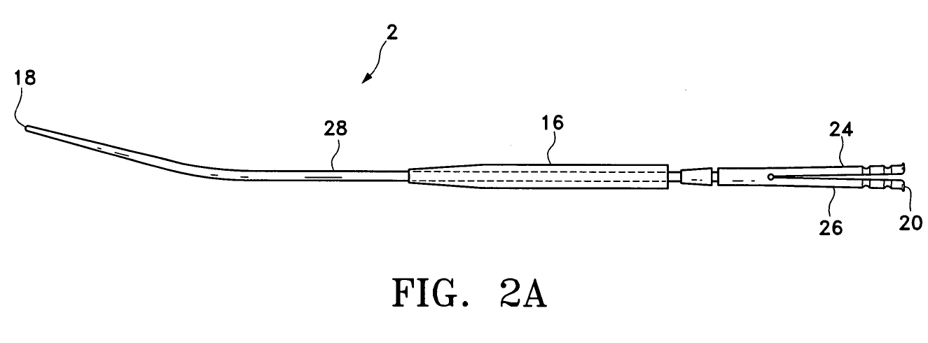

FIG. 2A illustrates another variation of the tunneling device 2 where the proximal end of the tunneling device is configured with two proximally projecting pieces 24, 26, having a curved inner profile for receiving a catheter. As shown, an oversleeve 16 is slidably positioned on the mid-shaft 28 of the tunneler. In this variation, the two proximally projecting pieces 24, 26, which form the fingers for trapping a catheter, is configured with a length “L” of 2.188 inches, an outer diameter “D1” at the distal end of 0.188 inches, an inner diameter “D2” of 0.168 inches, an outer diameter of “D3” at the proximal end of 0.230 inches, and the distance between the two fingers 24, 26 is “n” of 0.012 inches, as shown in FIG. 2B. The fingers 24, 26 are shown in a compressed position. A pair of grooves 28, 30 is provided to enhance the inner surface profile of the fingers. This may improve the contact between the fingers and the catheter and prevent the catheter from slipping out while the fingers are still in the compressed position. FIG. 2C shows the same sets of fingers 24, 26 in a relaxed and expanded position. FIG. 2D shows the oversleeve 16 which can be slid over the shaft of the tunneler and over the fingers to force the fingers to compress inward, thus trapping a catheter positioned between the fingers. As illustrated in FIG. 2E, the tunneling device 2 is shown with a portion of the catheter 22 secured within the proximal portion of the tunneling device 2.

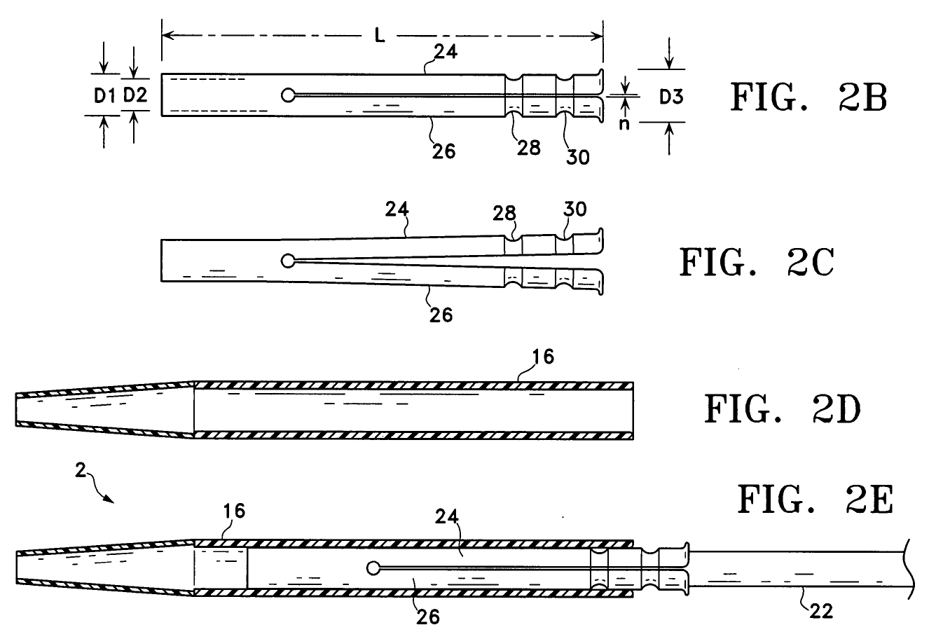

In another variation, the tunneling device is configured with a detachable gripping mechanism. In one example, the device comprises a collet that can be slidably removed from the tunneler body. As shown in FIG. 3A-3C, the tunneling device comprises a tunneler 32 with an elongated shaft, a collet 34 (i.e., compression sleeve) for trapping a catheter, and a sheath 36 (i.e., an oversleeve) for compressing the collet 36. In this example, the tunneler 32 comprises an elongated body with a tapered distal end 38 for insertion into a bodily tissue, as shown in FIG. 3A. The proximal end of the tunneler has a bulb or expanded profile 40 to prevent the collet 34 from sliding off from the proximal end 42. An optional raised profile 44 along the shaft of the tunneler may be provided to keep the collet 34 in position once it is slid over the tunneler 32. The collet 34 has a lumen 46 and two extending members 48, 50 for grabbing the distal portion of a catheter, as shown in FIG. 3B. FIG. 3C shows a sheath 36, which is configured to be slid over the collet 34 for compressing the extending members 48, 50 of the grabbing mechanism.

To assemble the tunneling device, the collet 34 is first slid over the tunneler 32 and advanced to the proximal portion 52 of the tunneler with the extending members 48, 50 of the collet 34 pointing toward the proximal end of the tunneler. The collet 34 is then slid over the raised profile 44 to secure the collet 34 into place. Alternatively, matching helical profiles may be provided on the outer surface of the tunneler' shaft and the inner lumen surface of the collet such that the collet may be screwed into place. Other locking mechanisms that are well known to one of ordinary skill in the art may also be applied to secure the collet. The sheath 36 is then slid over the tunneler 32 from the distal end 38 toward the proximal end 42. The proximal portion of the sheath 36 is then slid over the collet 34 to force the extending members 48, 50 of the collet 34 to compress inward. FIG. 3D illustrates the assembled three piece tunneling device with a catheter 54 positioned between the extending members 48, 50 of the collet 34 such that catheter 54 can be secured within the distal portion of the tunneling device.

Although it is preferred that the collet be configured to receive catheters of various sizes, one may also prepare a plurality of collets of various sizes to receive catheters of different diameters. For example, the size of the fingers and/or the radial expansion of the fingers may vary depending on the particular catheter or ranges of multiple catheters it is designed to support. In addition, differently sized collets may be configured for implementation on the same tunneler. For example, collets of different sizes may be configured with the same proximal lumen size for placement on the same tunneler. In this variation, the operator may select a specific collet size to use in the implant procedure, depending on the size of the catheter to be implanted. For example, a collection of collets may be provided with the inner lumen of each of the collets configured to surround a catheter of a specific French sizes ranging between about 1 French to about 30 French. Furthermore, one of ordinary skilled in the art would appreciate that the collet may be fabricated in various well known configurations.

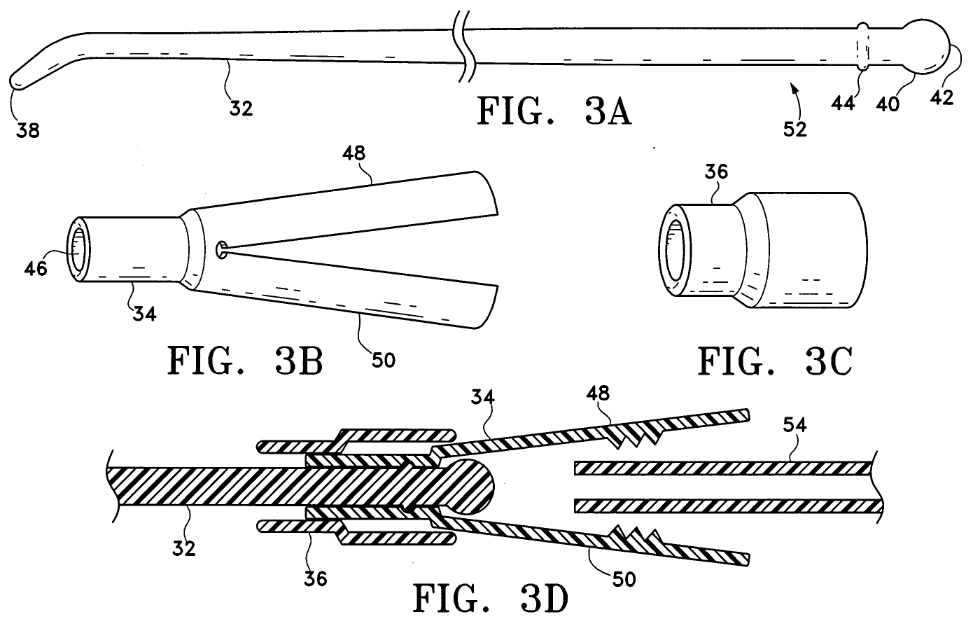

FIG. 4A illustrates another variation of the tunneling device 62, comprising a tunneler 64 with a compression sleeve 66 attached to the proximal end of the tunneler 64 and an oversleeve 68 slidably positioned along the shaft of the tunneler 64. The compression sleeve 66 may be detachable as shown in FIG. 4B. For example, one may prepare compression sleeve 66 of various sizes and/or varying inner surface profiles to accommodate catheters of different sizes and/or different tip configurations. Alternatively, the compression sleeve may be configured to accommodate catheters that fall within a range of sizes. The compression sleeve 66 may be configured to screw onto the proximal end of the tunneler 64 or otherwise attached to the proximal end of the tunneler through various locking or interconnecting mechanisms that are well known to one of ordinary skill in the art.

In the example shown in FIG. 4B, the compression sleeve 66 is configured to receive a dual lumen catheter with staggered distal ports. FIG. 4C shows the distal portion of a dual lumen catheter 70 positioned within the compression sleeve 66 while the compression sleeve 66 is in an expanded position. As the oversleeve 68 is pushed toward the proximal portion 72 of the tunneler 64, the fingers 74, 76 compress inward and trap the distal portion of the catheter 70. As shown in FIG. 4D, both distal exit ports 78, 80 of the catheter are secured within the tunneling device 62 when the oversleeve 68 is slid into place. This design may provide smoother tunneling of catheters having uneven profiles at the distal portion of the catheter. In this example, the edges of the proximal lumen opening 78, which may cause abrasion when inserted directly into an orifice of relatively small dimension, is covered by the fingers 74, 76. Furthermore, by covering the distal end and the lumen openings 78, 80 of the catheter, one may protect them from possible damage that may occur when the catheter is pulled through a tunneling channel within the tissue. Also, the inner surface of the fingers may be configured with ribs, teeth, or other surface profile 82 for preventing the catheter 70 from slipping out when the fingers 74, 76 are in the compressed position, as shown in FIG. 4E.

In light of the disclosure herein, one of ordinary skill in the art would appreciate that the tunneling device of the present invention may be configured to receive a catheter with various distal tip designs. For example, the gripping mechanism (e.g., collet, compression sleeve, extended fingers, etc.) may be configured to receive catheters with a staggered profile or having a plurality of lumens at the distal portion of the catheter (e.g., a Hickman® dual lumen catheter, BARD Access Systems, SLC, UT, etc.). Alternatively, the gripping mechanism may be configured to receive a catheter with a closed distal tip (e.g., Groshong catheter, etc.) by securing onto the distal portion of the catheter body. As described above, the tunneling device of the present invention may also be configured to receive a hemodialysis catheter having a split-tip configuration, such as the HemoSplit™ catheter, manufactured by Bard Access Systems, Inc. of Salt Lake City, Utah, which is disclosed in U.S. application Ser. No. 10/371,774, filed Feb. 21, 2003, and is incorporated by reference herein in its entirety. Due to the split-tip configuration of such hemodialysis catheters, it may be difficult to utilize a traditional catheter tunneler, which secures the catheter by attaching itself to the distal lumen of the catheter, as the tip section not attached to the tunneler will be left dangling and could suffer damage during a tunneling procedure. The tunneling device of the present invention may be configured with a large enough space between the fingers to receive both branches of the bifurcation, which forms the split-tip configuration, by compressing the distal portions of the two branches together within the proximal portion of the tunneling device. This may allow the physician to pull the split-tip catheter through the channel created by the tunneler without worrying about the possibility that the unsecured branch may get caught in the tissue or cause damage to the tissue during a tunneling procedure.

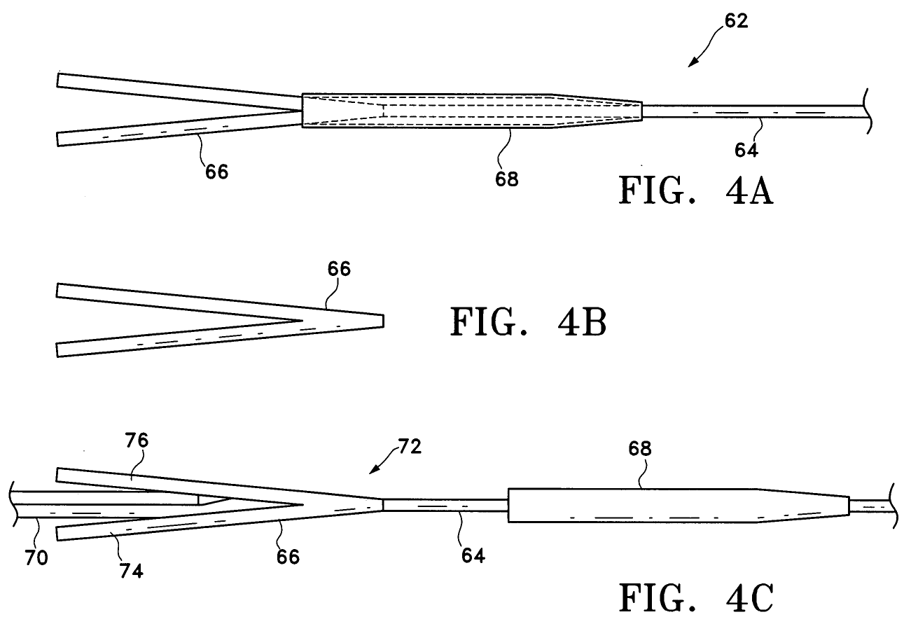

In another variation, the oversleeve may be configured with mechanisms for securement on the shaft of the tunneler. In addition, mechanisms may be provided to allow the operator to adjust the pressure applied on the catheter. For example, FIG. 5 illustrates one example where the inner lumen of the oversleeve 92 and the outer surface of the tunneler shaft 94, each is configured with matching helical grooves 96, 98, which allow incremental advancement of the oversleeve 92 on the shaft by rotation of the oversleeve 92. The incremental advancement of the oversleeve may allow the operator to gradually apply compression pressure on the compression sleeve 100. As one of ordinary skill in the art would appreciate, other well-known mechanical interfaces (e.g., grooves, notches, etc.) may also be applied to function as the interface between the oversleeve and the tunneler shaft. Furthermore, the oversleeve may comprise an interlayer (e.g., ribbon, spiral wrap plastic or metal spring), positioned in the inner lumen of the oversleeve to provide an interface between the oversleeve and the compression sleeve or the fingers of the collet. The interlayer may be flexible, compressible or otherwise pliable, to provide compression force on the fingers or to distribute pressure exerted by the oversleeve. The interlayer may also be configured such that a rotational force is required to remove the oversleeve from the distal portion of the tunneling device and allow the release of the compression fingers.

In another variation, the fingers and/or the proximal cavity may be adapted with padding or other soft and/or flexible interfaces for compressing and/or securing fragile materials, soft tissues or organs (e.g., tissue or organs harvested from the patient or a donor, etc.) for tunneling through the tissue in a patient. For example, the tunneling device may be configured for tunneling a vascular graft through the patient's body in order to position the vascular graft for implantation. In light of the disclosure herein, one of ordinary skill in the art would appreciate that the disclosed tunneling device may be fabricated in various sizes/dimensions and with various materials to accommodate different medical applications.

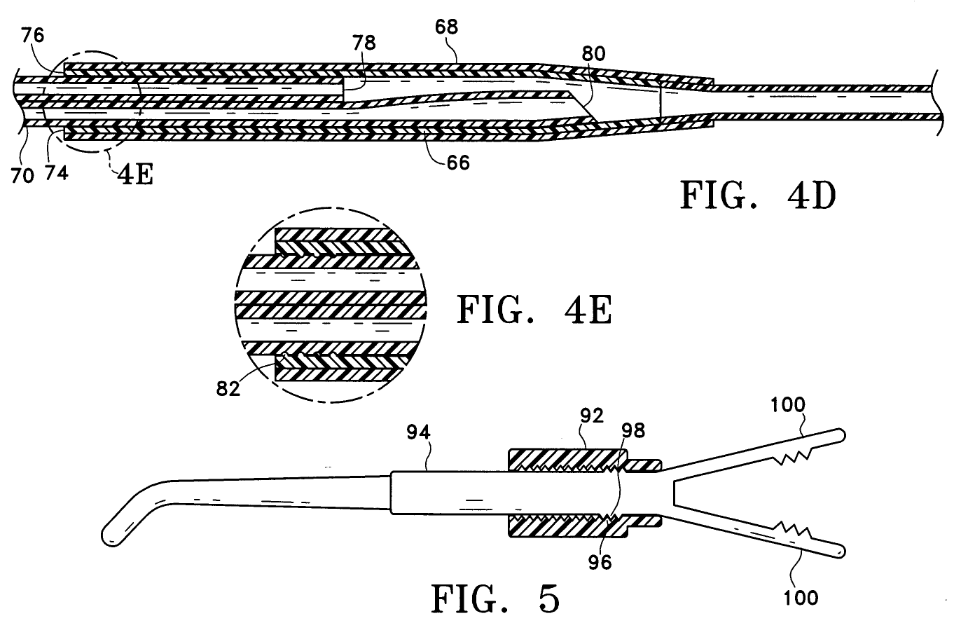

FIG. 6 illustrates another variation where an extended member 102 is provided at the proximal end of the tunneling device for insertion into the distal lumen 104 of the catheter 106. The extended member 102 may be provided to prevent the collapse and/or to provide support for the inner lumen of the catheter 106 such that the fingers 108, 110 of the gripping mechanism may achieve a better hold on the catheter 106. The extended member 102 may be a rod shaped structure positioned along the axis of the tunneling device. The diameter of the rod may be selected in accordance with the size of the inner lumen of the catheter it is designed support. Preferably, the diameter “d” of the rod is equal or less than the diameter “D” of the catheter's lumen. For example, one may select a diameter for the rod such that it can be easily inserted inside the catheter and still provide some support to counter the force form the compression of the FIGS. 108, 110. Optionally, surface profiles 114 (e.g., ribs, teeth, or grooves, etc.) may be provided on the fingers 108, 110 as shown in FIG. 6. In addition, surface profiles (e.g., ribs, teeth, grooves, etc.) may also be provided on the extended member 102. For example, grooves may be provided on the extended member 102 to match the ribs provided on the fingers 108, 110.

To utilize the tunneling device shown in FIG. 6, the operator first inserts the rod 102 into the distal opening of a catheter. The oversleeve 112 is then advanced toward the proximal end of the tunneling device (i.e., towards the catheter). The advancement of the oversleeve 112 forces the fingers 108, 110 to compress inward onto the body of the catheter. The rod 102, which is positioned inside the lumen of the catheter, may provide support to the lumen structure and counteract the compression force from the fingers 108, 110. As a result, a tighter grip on the catheter may be achieved. Alternatively, the extended member 102 may be provided for the sole purpose of positioning the catheter on the central axis of the tunneling device prior to compressing the fingers 108, 110 onto the catheter.

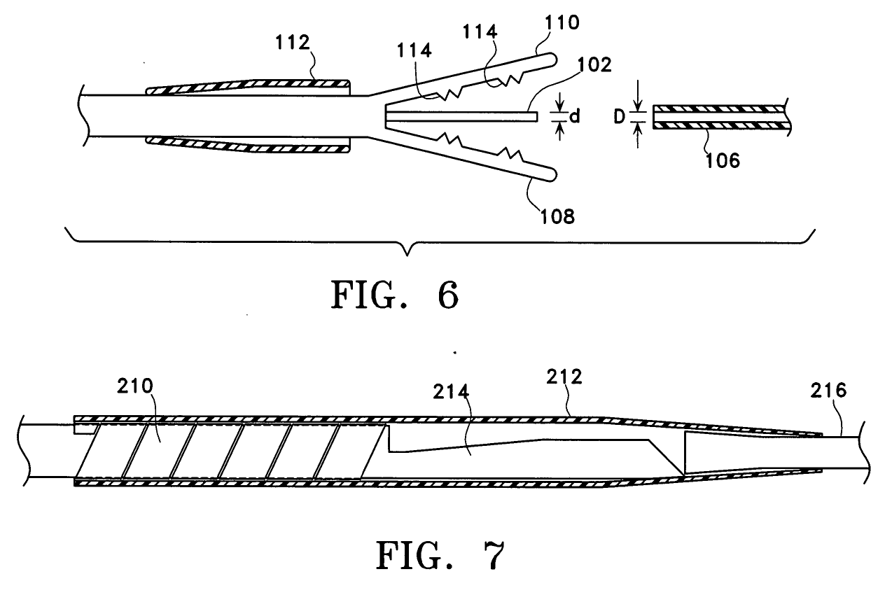

In yet another variation, the tunneling device may be configured without a collet. In one variation, the tunneling device comprises a sleeve 212 extending from the distal portion of a tunneler 216. Within the inner lumen of the sleeve a compressible material, such as a ribbon 210, is positioned to provide the compression force to secure the distal portion of the catheter 214 within the sleeve 212, as shown in FIG. 7. The ribbon 210 may be arranged such that a rotational force is needed to insert and retain the catheter 210 in the tunneling device. Other flexible, compressible or otherwise pliable materials may also be integrated within the sleeve to provide the necessary compression force for securing the distal portion of the catheter within the sleeve of the tunneling device. For example, a polymeric web, a polymer spring or a metal spring may be attached to the inner lumen of the sleeve to provide the compression force for grasping onto the body of the catheter.

As discussed earlier, the tunneling device may be implemented for tunneling various catheters or other elongated device/materials through a patient's bodily tissue. An example illustrating the implantation of a Hickman® catheter is described above. A first incision is made close to the patient's left nipple, and a second incision is made close to the left clavicle for accessing the subclavian vein. The distal tip portion of the Hickman® catheter is placed within the gripping mechanism of the tunneling device. An oversleeve is advanced toward the proximal end of the tunneling device to compress the gripping mechanism and secure the catheter within the gripping mechanism. The distal end of the tunneling device is then inserted into the first incision. The tunneler may be used to create a path between the first incision and the second incision. The tunneling device is pushed through the tissue to exit at the second incision. The tunneling device is pulled out of the second incision and, in the process, drags a portion of the catheter through the tunneled channel between the first and second incision. The oversleeve is pushed toward the distal end of the catheter to release the catheter from the gripping mechanism. A guidewire, introducer, and tear-away sheath may then be implemented to insert the distal portion of the catheter into the patient's circulatory system through the exposed subclavian vein. The physician may then advance the tip of the catheter towards the superior vena cava-right atrial junction. Once the catheter is put in place, sutures may be used to close the incisions sites as necessary.

In another variation, the catheter may be placed inside of a patient using the retrograde technique. In this approach, the distal tip of the catheter is first inserted into a vein in the patient's body. The proximal end of the catheter is then inserted into the proximal end of the tunneling device. The sleeve is then slid on, to force the gripping mechanism to secure a proximal portion of the catheter within the tunneling device. The tunneling device may then be used to tunneling the proximal end of the catheter under the skin to the final skin exit site.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

What is claimed as new and desired to be protected by Letters Patent of the United States is:

1. A tunneling device comprising:
an elongated body having a distal end and a proximal end, wherein said distal end is configured for tunneling through bodily tissue, and wherein said proximal end is configured for receiving a portion of a catheter; and
a compression mechanism deployed on said elongated body for securing said portion of said catheter within said proximal end of said elongated body.

2. The tunneling device according to claim 1, wherein said proximal end of said elongated body comprises a plurality of fingers extending away from said distal end of said elongated body.

3. The tunneling device according to claim 2, wherein said plurality of fingers are configured to expand outward from a central axis of said elongated body in a relaxed state.

4. The tunneling device according to claim 2, wherein said fingers form a receptacle for receiving the distal portion of said catheter.

5. The tunneling device according to claim 4, wherein said fingers are configured to receive a dual lumen catheter.

6. The tunneling device according to claim 5, wherein said dual lumen catheter comprises two staggered lumen openings at a distal portion thereof, said fingers being configured to enclose both of said staggered lumen openings.

7. The tunneling device according to claim 5, wherein said dual lumen catheter comprises a split-tip catheter having a plurality of tip sections at a distal end thereof, said fingers being configured to secure each of said plurality of tip sections.

8. The tunneling device according to claim 1, wherein said compression mechanism comprises an oversleeve slidably disposed on said elongated body, said oversleeve being configured for securing said portion of said catheter within said proximal end of said elongated body.

9. The tunneling device according to claim 2, wherein an inner surface of at least two of said fingers comprises protruding profiles for securing said catheter when said fingers are compressed inward toward a central axis of said elongated body.

10. The tunneling device according to claim 2, wherein said compression mechanism comprises an oversleeve slidably disposed on said elongated body, said oversleeve being configured to force said fingers to compress inward toward a central axis of said elongated body when said oversleeve is slid toward said proximal end of said elongated body.

11. The tunneling device according to claim 10, wherein an inner surface of each of said fingers comprises a protruding profile for securing said catheter when said fingers are compressed.

12. The tunneling device according to claim 11, wherein said protruding profile comprises a plurality of ribs.

13. The tunneling device according to claim 10, wherein said oversleeve comprises a tubular body.

14. The tunneling device according to claim 13, wherein said tubular body further comprises a surface profile on an inner surface thereof for compressing said fingers.

15. The tunneling device according to claim 14, wherein said surface profile comprises a ribbon disposed helically on said inner surface.

16. The tunneling device according to claim 10, wherein said oversleeve further comprises a spring positioned on an inner surface thereof.

17. The tunneling device according to claim 10, wherein said oversleeve further comprises a compression interface positioned in an inner lumen thereof.

18. A tunneling device comprising:
an elongated body configured for tunneling through bodily tissue;
a collet slidably disposed on said elongated body; and
a sheath slidably disposed on said elongated body for compressing said collet.

19. The tunneling device according to claim 18, wherein said collet comprises a compression sleeve, wherein said compression sleeve is configured to receive an object within a proximal end thereof after being positioned at a proximal end of said elongated body.

20. The tunneling device according to claim 18, wherein said collet comprises a tubular body with elongated members extending from a proximal end thereof, and wherein said sheath is configured to compress said elongated members of said tubular body toward a central axis of said elongated body when said sheath is slid over said tubular body.

21. The tunneling device according to claim 20, wherein said sheath comprises an oversleeve having a compression layer positioned in an inner lumen thereof.

22. The tunneling device according to claim 21, wherein said compression layer comprises a metal spring.

23. The tunneling device according to claim 18, wherein said collet is configured for receiving a distal portion or a proximal portion of a catheter.

24. The tunneling device according to claim 18, wherein said collet is configured for receiving a distal portion of a dual lumen catheter, said dual lumen catheter being configured with staggered distal lumen openings.

25. The tunneling device according to claim 18, wherein said collet is configured for securing a distal portion of a dual lumen catheter, wherein said dual lumen catheter is configured with a split distal end having at least two distal tips, and wherein said collet is configured to secure said distal tips of said catheter.

26. A method of assembling a tunneling device, comprising the steps of:
sliding a collet onto a distal end of a tunneler and toward a proximal end of said tunneler; and
sliding a sheath onto a distal end of said tunneler and toward the proximal end of said tunneler.

27. The method according to claim 26, wherein said collet is configured for receiving a portion of a catheter, further comprising the step of placing said portion of said catheter within said collet and sliding said sheath over said collet.

28. The method according to claim 26, wherein said collet is configured for receiving a distal portion of a dual lumen catheter, said dual lumen catheter being configured with staggered distal lumen openings, further comprising the step of placing said distal portion of said catheter within said collet and sliding said sheath over said collet.

29. The method according to claim 26, wherein said collet is configured for securing a plurality of tips of a split-tip catheter, further comprising the step of placing said tips of said catheter within said collet and sliding said sheath over said collet.

30. The method according to claim 26, wherein said collet is configured for receiving a portion of a catheter, and wherein said sheath further comprises a compression interface positioned in a lumen of said sheath, further comprising the step of placing said portion of said catheter within said collet and sliding said sheath over said collet.

31. A method of tunneling a catheter through bodily tissue, comprising the steps of:
inserting a portion of a catheter into a proximal portion of a tunneler;
securing said portion of said catheter to said tunneler; and
inserting a distal end of said tunneler into a first opening in a patient's body.

32. The method according to claim 31, wherein the step of inserting said portion of said catheter comprises inserting a portion of the catheter into a cavity surrounded by a plurality of fingers extending from a proximal end of said tunneler.

33. The method according to claim 32, wherein the step of securing said portion of said catheter comprises sliding an oversleeve over said plurality of fingers.

34. The method according to claim 31, wherein the step of securing said portion of said catheter comprises sliding a sheath towards the proximal end of said tunneler.

35. The method according to claim 31, further comprising the step of passing a portion of said catheter through said patient's body such that at least a portion of said catheter enters the patient's body through said first opening and exits said patient through a second opening in a patient's body.
